(19) United States
(12) Patent Application Publication
(10) Pub. No.: US 2023/0395202 A1
(43) Pub. Date: Dec. 7, 2023

(54) USING GLOBAL-SHAPE REPRESENTATIONS TO GENERATE A DEEP GENERATIVE MODEL

(71) Applicant: International Business Machines Corporation, Armonk, NY (US)

(72) Inventors: Payel Das, Yorktown Heights, NY (US); Yair Zvi Schiff, New York, NY (US); Enara C. Vijil, Millwood, NY (US); Samuel Chung Hoffman, New York, NY (US); Karthikeyan Natesan Ramamurthy, Pleasantville, NY (US)

(21) Appl. No.: 17/805,481

(22) Filed: Jun. 6, 2022

(51) Int. Cl. G16C 20/70; G16C 20/50

(52) U.S. Cl. CPC G16C 20/70; G16C 20/50

(57) ABSTRACT

Embodiments of the invention provide a computer-implemented method that includes applying input representations of a three-dimensional (3D) domain to a generative neural network (GNN); and using the GNN to form a generative model of the 3D domain based at least in part on the input representations. The input representations include a global-shape input representation of the 3D domain.

The following disclosure is submitted under 35 U.S.C. 102(b)(1)(A): DISCLOSURE: GRACE PERIOD DISCLOSURE; Schiff et al., “Augmenting Molecular Deep Generative Models with Topological Data Analysis Representations,” IEEE International Conference on Acoustics, Speech and Signal Processing, Submitted 8 Jun. 2021, Revised 15 Feb. 2022, pp. 3783-3787, arXiv:2106.04464v2.

### BACKGROUND

The present invention relates generally to programmable computers. More specifically, the present invention relates to programmable computer systems, computer-implemented methods, and computer program products operable to use global-shape representations to generate a deep generative model of a three-dimensional (3D) domain.

Predictive modeling is a type of machine learning problem that uses a model to make a prediction. The model is trained using a training dataset that includes multiple examples (or samples), each with input variables (X) and output class labels (y). The model is trained by showing the model examples of inputs, having it predict outputs, and correcting the model to make the outputs more like the expected outputs. The correction of the model is generally referred to as a supervised form of learning. In the predictive or supervised learning approach, the goal is to learn a mapping from inputs X to outputs y, given a labeled set of input-output pairs. Examples of supervised learning problems include classification and regression, and examples of supervised learning algorithms include logistic regression and random forest.

Unsupervised or descriptive learning is a type of machine learning problem where training data is not classified/labeled so must be developed through iterations of the machine learning algorithm(s). In unsupervised learning, the model is only given the input variables (X) and the problem does not have any output variables (y). The model is constructed by extracting or summarizing the patterns in the input data. There is no correction of the model as the model is not, in fact, predicting anything. This is a much less well-defined problem because the model is not told what kind of patterns to look for, and there is no obvious error metric to use unlike supervised learning where a comparison can be made between a prediction and the observed value.

Generative modeling is a type of unsupervised learning problem that automatically discovers and learns the regularities or patterns in input data in such a way that the model can be used to generate or output new examples that plausibly could have been drawn from the original dataset. Examples of unsupervised learning algorithms include generative algorithms such as auto-encoders (AE) (e.g., a variational AE (VAE)) and a generative adversarial network (GAN).

An autoencoder (AE) is a type of neural network based framework that includes an encoder-decoder pair, which learns how to efficiently compress and encode original data to a lower dimensional space known as “latent code” or “latent code space” then learns how to decompress the latent code to a representation of the original data (i.e., “reconstructed” original data) that is as close to the original data input as possible. The differences between the original data input and the reconstructed data output can be used to create encodings for expected output and vice versa. A VAE addresses the issue of non-regularized latent code in an AE and provides the generative capability to the entire latent code space. Similar to an AE, the encoder in a VAE outputs latent vectors, but instead of encoding an input as a single point, in VAE it is encoded as a distribution over the latent space. In standard VAE, a constraint is imposed on this latent distribution forcing it to be a standard normal. This constraint makes sure that the latent space is regularized or smoothed.

A GAN is a type of generative neural network that uses deep learning methods such as convolutional neural networks. A GAN trains a generative model by framing the problem as a supervised learning problem with two sub-models, namely a generator model and a discriminator model. The generator model is trained to generate new examples, and the discriminator model tries to classify examples as either real (i.e., from the domain) or fake (i.e., generated). The two models are trained together in an adversarial zero-sum game until the discriminator model is fooled about half the time, which means the generator model is generating plausible examples.

The successful performance of deep generative models in two-dimensional (2D) domains such as computer vision and natural language processing has led to attempts to create deep generative models in three-dimensional (3D) domains that include 3D shapes or geometries. In geometry, a 3D shape can be defined as a solid figure or an object or shape that has three dimensions, namely length, width, and height. Unlike 2D shapes, 3D shapes have thickness or depth. One example of a 3D domain is molecules. Most organic molecules, including all alkanes, are not planar but are instead characterized by 3D structures. Methane, for example, has the shape of a regular tetrahedron with carbon at the center and a hydrogen atom at each corner. Thus, the 3D arrangement of the atoms that constitute a molecule is known as molecular geometry and includes the general shape of the molecule as well as bond lengths, bond angles, torsional angles and any other geometrical parameters that determine the position of each atom. Generative models of molecules (i.e., generative molecular models) can be used in a variety of applications, including drug discovery and material design. How well the generative molecular model represents the molecule's 3D information determines how well the model represents (or reproduces) the molecule's functionality, including, for example, how the molecule binds to other elements and how the molecule expresses itself.

However, creating generative learning models that can correctly approximate the distribution of equilibrium 3D structures is challenging. Known approaches to creating such generative learning models that can correctly approximate the distribution of equilibrium 3D structures provide model input representations as strings and 2D graphs, which generally perform inefficiently and lack robustness in approximating 3D domains. The use of strings and 2D graphs as generative model input representations of 3D domains can also require specialized generative model architectures.

### SUMMARY

Embodiments of the invention provide a computer-implemented method that includes applying input representations of a three-dimensional (3D) domain to a generative neural network (GNN); and using the GNN to form a generative model of the 3D domain based at least in part on the input representations. The input representations include a global-shape input representation of the 3D domain. In accordance with aspects of the invention, including the global-shape input representation of the 3D domain among the input representations improves upon generative modeling approaches that rely on conventional point-level representations and point-by-point learning to create a model of the 3D domain. By including global-shape-based information when generating the generative model of the 3D domain, embodiments of the invention improve the resulting generative model's ability to represent or reproduce a wide range of the functionality of the 3D domain that is attributable to the 3D shapes or geometries that are present in the 3D domain.

In accordance with aspects of the invention, including the global-shape input representation of the 3D domain among the input representations improves upon generative modeling approaches that rely on conventional point-level representations and point-by-point learning to create a model of the 3D domain. By including global-shape-based information when generating the generative model of the 3D domain, embodiments of the invention improve the resulting generative model's ability to represent or reproduce a wide range of the functionality of the 3D domain that is attributable to the 3D shapes or geometries that are present in the 3D domain. In accordance with aspects of the invention, the global-shape input representations augment the local point-level representation, thereby augmenting the ability of the GNN to create a generative model that represents or reproduces a wide range of the functionality of the 3D domain that is attributable to the 3D shapes or geometries that are present in the 3D domain. In accordance with aspects of the invention, including the characteristics of the 3D domain among the input representations improve the resulting generative model's ability to represent or reproduce a wide range of the functionality of the 3D domain that is attributable to the characteristics that are present in the 3D domain.

In addition to one or more of the features described above, or as an alternative to any of the foregoing embodiments, the global-shape input representation of the 3D domain includes a persistence image. In accordance with aspects of the invention, the persistence image is a vector-based version of the global-shape input representations that can more efficiently and effectively be processed and analyzed by the GNN when forming the generative model of the 3D domain.

In addition to one or more of the features described above, or as an alternative to any of the foregoing embodiments, the input representations further include a local point-level input representation of the 3D domain. In accordance with aspects of the invention, the global-shape input representations augment the local point-level representation, thereby augmenting the ability of the GNN to create a generative model that represents or reproduces a wide range of the functionality of the 3D domain that is attributable to the 3D shapes or geometries that are present in the 3D domain.

In addition to one or more of the features described above, or as an alternative to any of the foregoing embodiments, using the GNN to form the generative model of the 3D domain includes encoding, using the GNN, the input representations to generate latent code; decoding, using the GNN, the latent code to generate a reconstructed version of the input representations; and generating, using the GNN, a reconstruction loss based at least in part on the reconstructed version of the input representations.

In addition to one or more of the features described above, or as an alternative to any of the foregoing embodiments, the local point-level input representation includes a string input representation; and the global-shape input representation includes a persistence image input representation.

In addition to one or more of the features described above, or as an alternative to any of the foregoing embodiments, the input representations further includes an input representation of a characteristic of the 3D domain; the global-shape input representation of the 3D domain is represented as a first parameter of a multi-parameter persistence image; and the input representation of the characteristic of the 3D domain is represented as a second parameter of the multi-parameter persistence image. In accordance with aspects of the invention, including the characteristics of the 3D domain among the input representations improve the resulting generative model's ability to represent or reproduce a wide range of the functionality of the 3D domain that is attributable to the characteristics that are present in the 3D domain. In accordance with aspects of the invention, using the multi-parameter persistence image reduces the GNN elements (e.g., encoder-decoder stages) required to process the input representations.

In addition to one or more of the features described above, or as an alternative to any of the foregoing embodiments, embodiments of the invention further provide computer systems and computer program products having substantially the same features as the above-described computer-implemented methods.

Embodiments of the invention provide a computer system that includes a memory and a processor communicatively coupled to the memory. The processor is operable to form a generative model of a three-dimensional (3D) domain by performing operations that include encoding, using a GNN, input representations to generate latent code. The input representations include a string input representation of the 3D domain, along with and a 3D coordinates representation of the 3D domain. The operations further include decoding, using the GNN, the latent code to generate a reconstructed version of the input representations; and generating, using the GNN, a reconstruction loss based at least in part on the reconstructed version of the input representations. In accordance with aspects of the invention, including 3D coordinates representation of the 3D domain among the input representations improves upon generative modeling approaches that rely on the string input representations and point-by-point learning to create a model of the 3D domain. By including the 3D coordinates-based information when generating the generative model of the 3D domain, embodiments of the invention improve the resulting generative model's ability to represent or reproduce a wide range of the functionality of the 3D domain that is attributable to the 3D shapes or geometries that are present in the 3D domain.

In addition to one or more of the features described above, or as an alternative to any of the foregoing embodiments, the input representations further include an input representation of a characteristic of the 3D domain. The 3D coordinates representation of the 3D domain is represented as a first parameter of a multi-parameter persistence image; and the input representation of the characteristic of the 3D domain is represented as a second parameter of the multi-parameter persistence image. In accordance with aspects of the invention, including the characteristics of the 3D domain among the input representations improve the resulting generative model's ability to represent or reproduce a wide range of the functionality of the 3D domain that is attributable to the characteristics that are present in the 3D domain. In accordance with aspects of the invention, using the multi-parameter persistence image reduces the GNN elements (e.g., encoder-decoder stages) required to process the input representations.

In addition to one or more of the features described above, or as an alternative to any of the foregoing embodiments, embodiments of the invention further provide computer-implemented methods and computer program products having substantially the same features as the above-described computer system.

Embodiments of the invention provide a computer system that includes a memory and a processor communicatively coupled to the memory. The processor is operable to form a generative model of a 3D domain by performing operations that include encoding, using a GNN, input representations to generate latent code. The input representations include a string representation of the 3D domain; a 3D coordinates representation of the 3D domain; and an input representation of a characteristic of the 3D domain. The operations further include decoding, using the GNN, the latent code to generate a reconstructed version of the input representations; and generating, using the GNN, a reconstruction loss based at least in part on the reconstructed version of the input representations. In accordance with aspects of the invention, including the 3D coordinates representation of the 3D domain among the input representations improves upon generative modeling approaches that rely on string input representations and point-by-point learning to create a model of the 3D domain. By including the 3D coordinates information when generating the generative model of the 3D domain, embodiments of the invention improve the resulting generative model's ability to represent or reproduce a wide range of the functionality of the 3D domain that is attributable to the 3D shapes or geometries that are present in the 3D domain. In accordance with aspects of the invention, the 3D coordinates representations augment the string representation, thereby augmenting the ability of the GNN to create a generative model that represents or reproduces a wide range of the functionality of the 3D domain that is attributable to the 3D shapes or geometries that are present in the 3D domain. In accordance with aspects of the invention, including the characteristics of the 3D domain among the input representations improve the resulting generative model's ability to represent or reproduce a wide range of the functionality of the 3D domain that is attributable to the characteristics that are present in the 3D domain.

In addition to one or more of the features described above, or as an alternative to any of the foregoing embodiments, the 3D domain includes a molecule; and the characteristic of the molecule is selected from the group consisting of an atomic charge and an atomic weight.

In addition to one or more of the features described above, or as an alternative to any of the foregoing embodiments, embodiments of the invention further provide computer-implemented methods and computer program products having substantially the same features as the above-described computer system.

Additional features and advantages are realized through the techniques described herein. Other embodiments and aspects are described in detail herein. For a better understanding, refer to the description and to the drawings.

### BRIEF DESCRIPTION OF THE DRAWINGS

The subject matter which is regarded as the present invention is particularly pointed out and distinctly claimed in the claims at the conclusion of the specification. The foregoing and other features and advantages are apparent from the following detailed description taken in conjunction with the accompanying drawings in which:

FIG. 1A depicts a simplified diagram of a three-dimensional (3D) domain that can be modeled using aspects of the invention;

FIG. 1B depicts a diagram illustrating an example of 3D domain surface shape of the 3D domain shown in FIG. 1A;

FIG. 2 depicts a system in accordance with embodiments of the invention;

FIG. 3 depicts an example implementation of a generative network in accordance with aspects of the invention;

FIG. 4A depicts a simplified diagram illustrating a local point-level input representation in accordance with aspects of the invention;

FIG. 4B depicts a simplified diagram illustrating a global-shape input representation in accordance with aspects of the invention;

FIG. 4C depicts a simplified diagram illustrating a node-level characteristic input representation in accordance with aspects of the invention;

FIG. 5A depicts a flow diagram illustrating a methodology in accordance with aspects of the invention;

FIG. 5B depicts a flow diagram illustrating a methodology in accordance with aspects of the invention;

FIG. 5C depicts a flow diagram illustrating a methodology in accordance with aspects of the invention;

FIG. 5D depicts a flow diagram illustrating a methodology in accordance with aspects of the invention;

FIG. 6 depicts a simplified diagram of a 3D molecular domain that can be modeled using aspects of the invention;

FIG. 7 depicts a simplified molecular input line entry system (SMILES) molecular representation capable of being utilized in accordance with aspects of the invention;

FIG. 8 depicts diagrams illustrating how topological data analysis (TDA) can be used to generate a persistence image in accordance with aspects of the invention;

FIG. 9 depicts a system in accordance with aspects of the invention;

FIG. 10 depicts objective functions executed by the system shown in FIG. 9 in accordance with aspects of the invention;

FIG. 11 depicts a programmable computer system capable of implementing aspects of the invention;

FIG. 12 depicts a cloud computing environment according to embodiments of the present invention; and

FIG. 13 depicts abstraction model layers according to an embodiment of the present invention.

In the accompanying figures and following detailed description of the disclosed embodiments, the various elements illustrated in the figures are provided with three-digit reference numbers. The leftmost digit of each reference number corresponds to the figure in which its element is first illustrated.

### DETAILED DESCRIPTION

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Many of the functional units described in this specification are illustrated as logical blocks such as encoders, decoders, classifiers, discriminators, modules, processors, and the like. Embodiments of the invention apply to a wide variety of implementations of the logical blocks described herein. For example, a given logical block can be implemented as a hardware circuit operable to include custom VLSI circuits or gate arrays, as well as off-the-shelf semiconductors such as logic chips, transistors, or other discrete components. The logical blocks can also be implemented in programmable hardware devices such as field programmable gate arrays, programmable array logic, programmable logic devices, and the like. The logical blocks can also be implemented in software for execution by various types of processors. Some logical blocks described herein can be implemented as one or more physical or logical blocks of computer instructions which can, for instance, be organized as an object, procedure, or function. The executables of a logical block described herein need not be physically located together but can include disparate instructions stored in different locations which, when joined logically together, include the logical block and achieve the stated purpose for the logical block.

Turning now to a more detailed description of technologies related to aspects of the invention, a biological neuron provides a template for the various types of artificial neural network architectures. In general, a biological neuron has pathways that connect it to upstream inputs, downstream outputs, and downstream “other” neurons. Each biological neuron sends and receives electrical impulses through pathways. The nature of these electrical impulses and how they are processed in the biological neuron are primarily responsible for overall brain functionality. The pathway connections between the biological neurons can be strong or weak. When the neuron receives input impulses, the neuron processes the input according to the neuron's function and sends the result of the function on a pathway to downstream outputs and/or on a pathway to downstream “other” neurons. A normal adult human brain includes about one hundred billion interconnected neurons.

In artificial neural networks, the biological neuron is modeled as a node having a mathematical function, f(x). Each node in the neural network receives electrical signals from inputs over one of multiple pathways, multiplies each input by the strength of its respective connection pathway, takes a sum of the inputs, passes the sum through a function (f(x)) of the node, and generates a result, which may be a final output or an input to another node, or both. Weak input signals are multiplied by a very small connection strength number, so the impact of a weak input signal on the function is very low. Similarly, strong input signals are multiplied by a higher connection strength number, so the impact of a strong input signal on the function is larger. The function f(x) is a design choice, and a variety of functions can be used. A suitable design choice for f(x) is the hyperbolic tangent function, which takes the function of the previous sum and outputs a number between minus one and plus one.

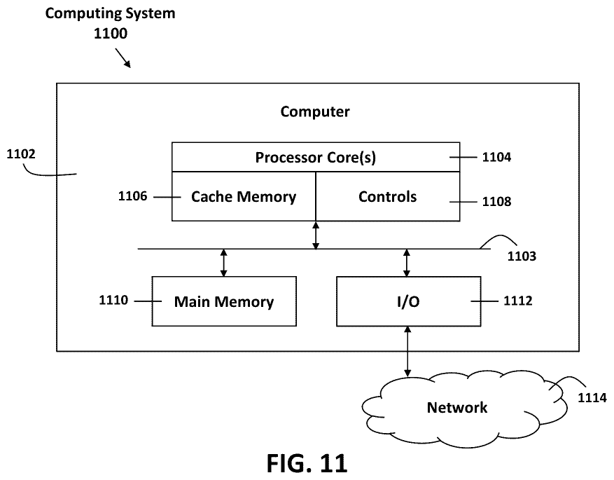

In general, neural networks can be implemented as a set of algorithms (e.g., machine learning algorithms) running on a programmable computer (e.g., computer systems 1100 shown in FIG. 11). In some instances, neural networks are implemented on an electronic neuromorphic machine (e.g., the IBM®/DARPA SyNAPSE computer chip) that attempts to create connections between processing elements that are substantially the functional equivalent of the synapse connections between brain neurons. In either implementation, neural networks incorporate knowledge from a variety of disciplines, including neurophysiology, cognitive science/psychology, physics (statistical mechanics), control theory, computer science, artificial intelligence, statistics/mathematics, pattern recognition, computer vision, parallel processing and hardware (e.g., digital/analog/VLSI/optical).

The basic function of a neural network is to recognize patterns by interpreting sensory data through a kind of machine perception. Real-world data in its native form (e.g., images, sound, text, or time series data) is converted to a numerical form (e.g., a vector having magnitude and direction) that can be understood and manipulated by a computer. The neural network creates a “model” that is “trained” by performing multiple iterations of learning-based analysis on the real-world data vectors until patterns (or relationships) contained in the real-world data vectors are uncovered and learned. The patterns uncovered/learned by the model of the neural network can be used to perform a variety of tasks. The learning or training performed by the machine learning algorithms on the model can be supervised, unsupervised, or a hybrid that includes aspects of supervised and unsupervised learning. Supervised learning is when training data is already available and classified/labeled. Unsupervised learning is when training data is not classified/labeled so must be developed through iterations of the neural network and the machine learning algorithms. Unsupervised learning can utilize additional learning/training methods including, for example, clustering, anomaly detection, neural networks, deep learning, and the like.

Turning now to an overview of aspects of the invention, embodiments of the invention provide programmable computer systems, computer-implemented methods, and computer program products operable to efficiently use three-dimensional (3D) global-shape representations to train or generate a deep generative model of a 3D domain. Known approaches to creating a generative model of any domain utilize machine learning techniques, which analyze local or localized node-level or point-level input representations of the domain. Each point-level input representation is local or localized in that it provides information that is valid only for a relatively small radius around the relevant point (or node). Accordingly, known approaches to machine learning configure machine learning algorithms to “learn” patterns in point-level input representations of a domain by moving point-by-point (or node-by-node) through the input representations regardless of whether the domain being modeled is 2D or 3D.

Local point-level domain representations are sufficient for modeling 2D domains. However, creating generative learning models that can correctly approximate the distribution of equilibrium 3D structures using local point-level representations and point-by-point learning is challenging. Known local point-level representations such as strings and 2D graphs generally perform inefficiently and lack robustness in approximating 3D domains. The use of strings and 2D graphs as generative model input representations of 3D domains can also require specialized generative model architectures.

Embodiments of the invention address the shortcomings of using local point-level representations and point-by-point learning to model 3D domains by providing programmable computer systems, computer-implemented methods, and computer program products operable to use 3D global-shape input representations to generate a deep generative model of a 3D domain. In contrast to local point-level input representations and point-by-point learning, global-shape input representations provide information that is valid for the entire domain, which is particularly beneficial when the domain is a 3D domain that include 3D shapes or geometries. In geometry, a 3D shape can be defined as a solid figure or an object or shape that has three dimensions, namely length, width, and height. Unlike 2D shapes, 3D shapes have thickness or depth. One example of a 3D domain is molecules. Most organic molecules, including all alkanes, are not planar but are instead characterized by 3D structures. Methane, for example, has the shape of a regular tetrahedron with carbon at the center and a hydrogen atom at each corner. Thus, the 3D arrangement of the atoms that constitute a molecule is known as molecular geometry and includes the general shape of the molecule as well as bond lengths, bond angles, torsional angles and any other geometrical parameters that determine the position of each atom.

It has been discovered by the inventors of the embodiments of the present invention that using global-shape input representations that provide information that is valid for the entire 3D domain to create generative models of the 3D domain improves how well the generative model represents the domain's 3D information, which improves how well the model represents (or reproduces) the 3D domain's functionality. As a non-limiting example, where the 3D domain is the 3D structure of a molecule, using global-shape input representations that provide information that is valid for the entire 3D molecular domain to create a generative molecular model of the 3D molecular domain improves how well the generative molecular model represents the molecule's 3D information. How well the generative molecular model represents the molecule's 3D information determines how well the model represents (or reproduces) the molecule's functionality, including, for example, how the molecule binds to other elements and how the molecule expresses itself. Accordingly, embodiments of the invention improve the performance of generative molecular models in in a variety of applications, including drug discovery and material design.

In embodiments of the invention, the global-shape input representations of a given 3D domain are developed by leveraging topological data analysis (TDA) to extract translation, rotation, and node permutation invariant global 3D information about 3D domains. In a non-limiting example, the 3D domain is a molecule, and embodiments of the invention enhance the spatial information content of a molecular generative model by encoding TDA representations of the molecule. Using noise-robust topological summaries of molecules, TDA provides 3D geometric information and can incorporate the influence of other node-level characteristics such as atomic charge, atomic weight, and the like. TDA representations have low computational complexity because they can be pre-computed. Additionally, TDA representations are a good examples of a global-shape representation because they incorporate global topological information, which cannot be captured by 2D point-level (or node-level) representations such as molecular graphs or point clouds. TDA representations are naturally invariant to translations and rotations of molecules and are also equivariant to scaling of distances between atoms of a molecule and permutation of the node order in the molecular graph (assuming that topological summaries are derived for functions that do not depend on node order, such as distance between atoms). Thus, TDA representations are robust to the coordinate system used to represent the molecular parameters. They are not overly sensitive to noise in the input parameters, which can help in generalization in real-world scenarios where molecules exhibit conformational dynamics. Features extracted from the distance matrix or graph Laplacians of the point cloud representation of atoms do not capture higher level topological features; may not provide scale equivariance and invariance to node order; and have no structured way of incorporating multiple functions, e.g., inter-atom distances and charges.

In some embodiments of the invention, input representations of a 3D domain are applied to a generative neural network (GNN), and the GNN uses the input representations to form a generative model of the 3D domain. The input representations include a global-shape input representation of the 3D domain. In accordance with aspects of the invention, including the global-shape input representation of the 3D domain among the input representations improves upon generative modeling approaches that rely on, for example, conventional point-level input representations and point-by-point learning to create a model of the 3D domain. By including global-shape-based information when generating the generative model of the 3D domain, embodiments of the invention improve the resulting generative model's ability to represent or reproduce a wide range of the functionality of the 3D domain that is attributable to the 3D shapes or geometries that are present in the 3D domain.

In some embodiments of the invention, the GNN is implemented as a generative adversarial network (GAN). The GAN is operable to train the 3D generative model by framing the problem as a supervised learning problem with two sub-models, namely a generator model and a discriminator model. The generator model is trained to generate new examples based on the input representations, and the discriminator model tries to classify examples as either real (i.e., from the domain) or fake (i.e., generated). The two models are trained together in an adversarial zero-sum game until the discriminator model is fooled about half the time, which means the generator model is generating plausible examples.

In some embodiments of the invention, the GNN is implemented as a variational auto-encoder (VAE) having an encoder-decoder path that is operable to include the processing of global-shape input representations of the 3D domain. Reconstruction losses associated with the encoder-decoder path are used to train a generative model of the 3D domain. In some embodiments of the invention, the training process is further enhanced by the encoder-decoder path being operable to process node-level characteristics of the 3D domain.

In some embodiments of the invention, the GNN is implemented as a VAE having multiple encoder-decoder paths. A first encoder-decoder path is used to process local point-level input representations of a 3D domain; and a second encoder-decoder path is used to process global-shape input representations of the 3D domain. Reconstruction losses associated with the first and second encoder-decoder paths are used to train a generative model of the 3D domain. In some embodiments of the invention, the training process is further enhanced by providing a third encoder-decoder path that processes node-level characteristics of the 3D domain, and the reconstruction losses associated with the first, second and third encoder-decoder paths are used to train the generative model of the 3D domain.

In some embodiments of the invention, the global-shape input representation of the 3D domain includes a persistence image input representation. In accordance with aspects of the invention, the persistence image is a vector-based version of the global-shape input representations that can more efficiently and effectively be processed and analyzed by the GNN when forming the generative model of the 3D domain.

In some embodiments of the invention, the global-shape input representation of the 3D domain is represented as a first parameter of a multi-parameter persistence image; and a characteristic of the 3D domain is represented as a second parameter of the multi-parameter persistence image. In accordance with aspects of the invention, including the characteristics of the 3D domain among the input representations improve the resulting generative model's ability to represent or reproduce a wide range of the functionality of the 3D domain that is attributable to the characteristics that are present in the 3D domain. In accordance with aspects of the invention, using the multi-parameter persistence image reduces the GNN elements (e.g., encoder-decoder stages) required to process the input representations.

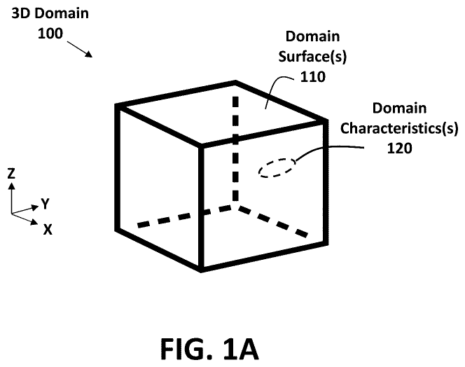

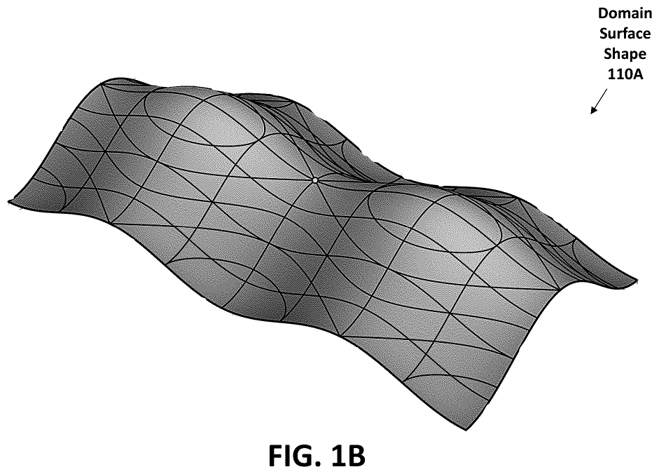

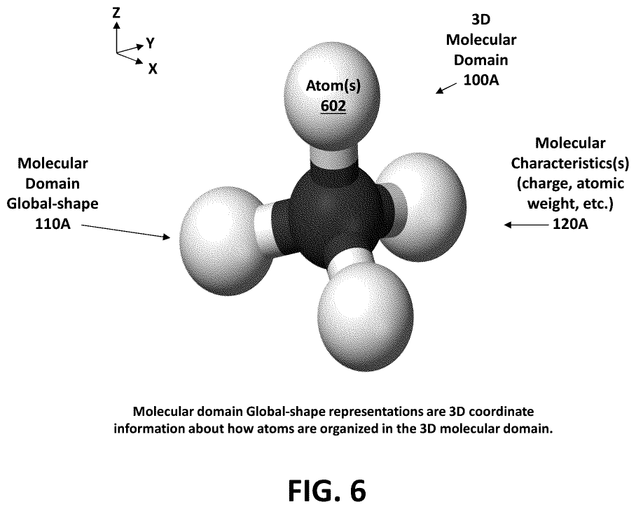

Turning now to a more detailed description of aspects of the invention, FIG. 1A depicts a simplified diagram of a generic 3D domain 100 that can be modeled using aspects of the invention. The 3D domain 100 is generic in that it represents any 3D domain having any size, shape, or combination of shapes. The 3D domain 100 is not planar but is instead characterized by 3D structures that include 3D shapes or geometries. In geometry, a 3D shape can be defined as a solid figure or an object or shape that has three dimensions, namely length, width, and height. Unlike 2D shapes, 3D shapes have thickness or depth. One example implementation of the 3D domain 100 is a 3D molecular domain 100A (shown in FIG. 6). As shown in FIG. 1A, the 3D domain 100 includes a plurality of domain surfaces 110 and a plurality of domain characteristics 120. In accordance with aspects of the invention, the domain surfaces 110 can be implemented as domain surfaces 110A (shown in FIG. 1B) having 3D topologies.

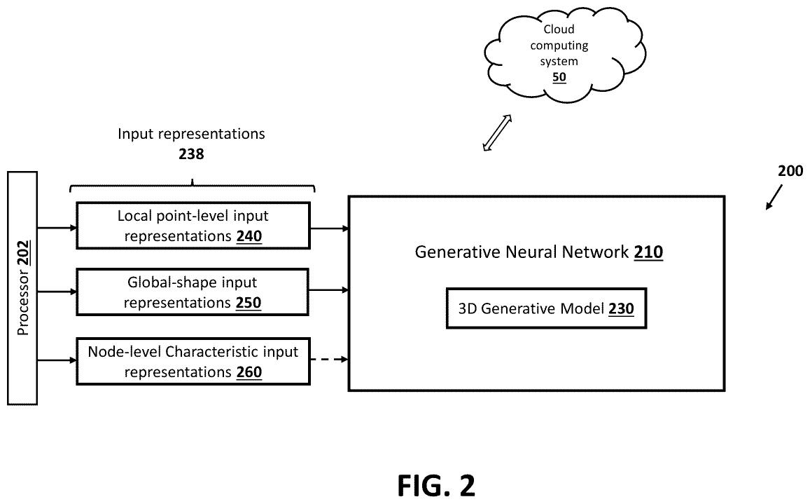

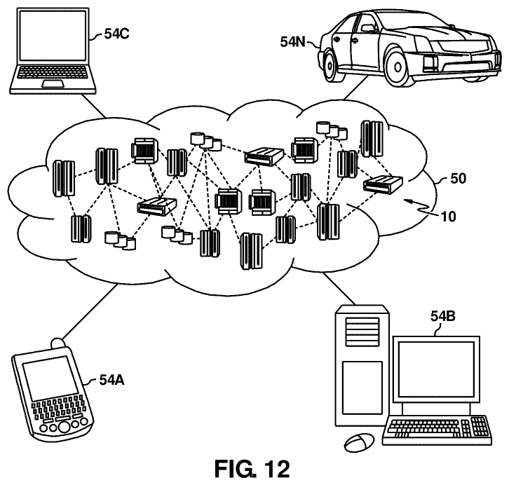

FIG. 2 depicts a system 200 operable to generate or train a 3D generative model 230 of a 3D domain (e.g., 3D domain 100 shown in FIG. 1A) in accordance with embodiments of the invention. As shown, the system 200 includes a GNN 210 and a processor 202 configured and arranged as shown. The processor 202 is operable to generate input representations 238 and provide the same to the GNN 210. In some embodiments of the invention, the input representations 238 include any combination of one or more of the local point-level input representations 240, global-shape input representations 250, and node-level characteristic input representations 260. Cloud computing system 50 is in wired or wireless electronic communication with one or all of the elements of the system 200. Cloud computing system 50 can supplement, support, or replace some or all of the functionality of the elements of the system 200. Additionally, some or all of the functionality of the elements that form the system 200 can be implemented as a node 10 (shown in FIG. 12) of cloud computing system 50.

The various components/modules of the system 200 shown in FIG. 2 are depicted separately for ease of illustration and explanation. In embodiments of the invention, the functions performed by the various components/modules of the system 200 can be distributed differently than shown. For example, in some embodiments of the invention, the generative neural network 210 could be integrated into the processor 202 and vice versa.

In embodiments of the invention, the input representation(s) 238 of a 3D domain (e.g., 3D domain 100 shown in FIG. 1A) are applied singularly or in any combination to the GNN 210, and the GNN 210 uses the input representations 238 to form the 3D generative model 230 of the 3D domain. As noted, the input representations 238 include the global-shape input representation 250 of the 3D domain. In accordance with aspects of the invention, including the global-shape input representation 250 of the 3D domain among the input representations 238 improves upon generative modeling approaches that rely on conventional point-level representations and point-by-point learning to create a model of the 3D domain. By including global-shape input representations 250 when generating the 3D generative model 230 of the 3D domain, embodiments of the invention improve the ability of the resulting 3D generative model 230 to represent or reproduce a wide range of the functionality of the 3D domain that is attributable to the 3D shapes or geometries that are present in the 3D domain.

In some embodiments of the invention, the GNN 210 is implemented as a GAN having a discriminator stage and a generator stage. In some embodiments of the invention, the GNN 210 is implemented as a VAE having an encoder-decoder path that is operable to include processing of the global-shape input representations 250 of the 3D domain. Reconstruction losses associated with the encoder-decoder path are used to train the 3D generative model 230 of the 3D domain. In some embodiments of the invention, the training process is further enhanced by the encoder-decoder path being operable to process the node-level characteristics input representations 260 of the 3D domain. In some embodiments of the invention, the GNN 210 is implemented as a VAE having multiple encoder-decoder paths. In embodiments of the invention where the GNN 210 is implemented as a VAE having multiple encoder-decoder paths, a first encoder-decoder path is used to process local point-level input representations 240 of the 3D domain; and a second encoder-decoder path is used to process global-shape input representations 250 of the 3D domain. Reconstruction losses associated with the first and second encoder-decoder paths are used to train the 3D generative model 250 of the 3D domain. In some embodiments of the invention, the training process is further enhanced by providing a third encoder-decoder path that processes the node-level characteristic input representations 260 of the 3D domain, and the reconstruction losses associated with the first, second and third encoder-decoder paths are used to train the 3D generative model 230 of the 3D domain.

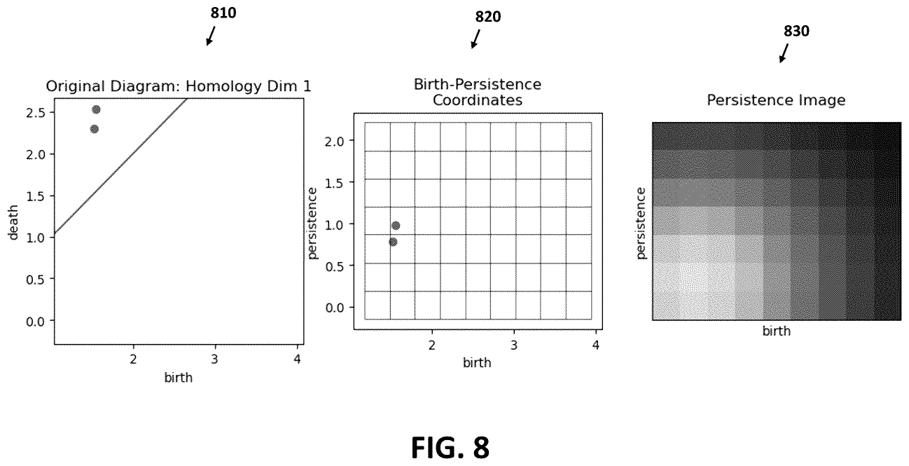

In some embodiments of the invention, the global-shape input representation 250 of the 3D domain includes a persistence image (e.g., persistence image 830 shown in FIG. 8). In accordance with aspects of the invention, the persistence image is a vector-based version of the global-shape input representation 250 that can more efficiently and effectively be processed and analyzed by the GNN 210 when forming the 3D generative model 230 of the 3D domain.

In some embodiments of the invention, the global-shape input representation 250 of the 3D domain is represented as a first parameter of a multi-parameter persistence image; and the node-level characteristic input representation 260 of the 3D domain is represented as a second parameter of the multi-parameter persistence image. In accordance with aspects of the invention, including the node-level characteristic input representation 260 of the 3D domain among the input representations 238 improve the ability of the resulting 3D generative model 230 to represent or reproduce a wide range of the functionality of the 3D domain that is attributable to the characteristics that are present in the 3D domain. In accordance with aspects of the invention, using the multi-parameter persistence image reduces the GNN elements (e.g., encoder-decoder stages) required to process the input representations 238.

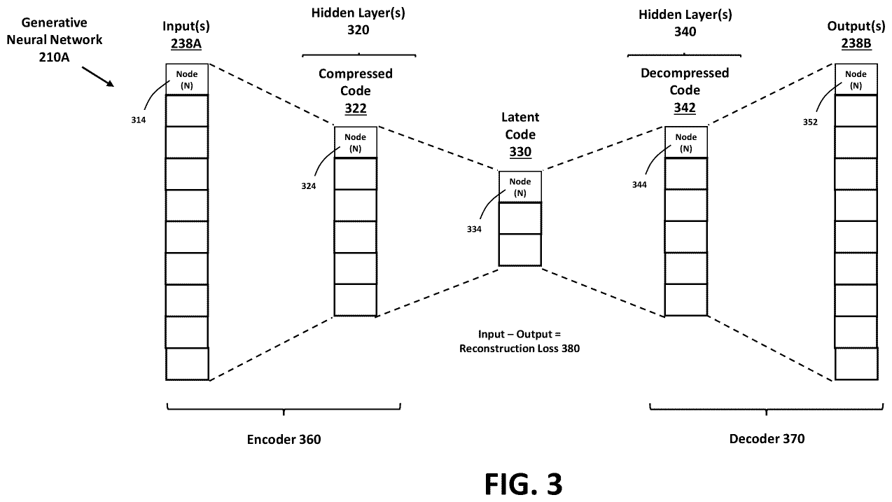

FIG. 3 depicts a GNN 210B in accordance with aspects of the invention. The GNN 210A is an example implementation of the GNN 210 (shown in FIG. 2). In accordance with embodiments of the invention, the GNN 210A includes an encoder stage 360, a latent code stage 330, and a decoder stage 370. The encoder stage 360 receives inputs 238A and performs multiple successive compressions or encodings to generate compressed code 322 in hidden layers 320. Although only one instance of compressed code 322 is shown, any number of successive compressions or encodings can be employed until a desired lower dimension space for the latent code 330 is reached.

The latent code 330 is provided to a decoder stage 370 where it is decompressed through successive decompressions to generate decompressed code 342 in hidden layer 340. Although only one instance of decompressed code 342 is shown, any number of successive decompressions or decodings can be employed until a reconstructed version of the inputs 238A is produced at the outputs 238B. The inputs 238A minus the outputs 238B represent a reconstruction loss 380. In a VAE implementation of the GNN 210A, the training is “regularized” to avoid overfitting and ensure that the latent code 330 has good properties that enable generative process. A VAE addresses the issue of non-regularized latent code and provides the generative capability to the entire latent code space 330. Similar to an AE, the encoder in a VAE outputs latent vectors, but instead of outputting the vectors in the latent code space 330, the encoder of a VAE outputs parameters of a pre-defined distribution in the latent code space 330 for every input 238A. The VAE then imposes a constraint on this latent distribution forcing it to be a normal or smooth distribution. This constraint makes sure that the latent code 330 is regularized or smoothed.

The inputs 238A, compressed code 322, latent code 330, decompressed code 342, and outputs 238B are each represented as a first series of nodes (N) 314, a second series of nodes (N) 324, a third series of nodes (N) 334, a fourth series of nodes (N) 344, and a fifth series of nodes (N) 352, respectively. In accordance with embodiments of the invention, the inputs 238A and the outputs 238B can each be presented as any combination of one or more of the input representations 238 shown in FIG. 2.

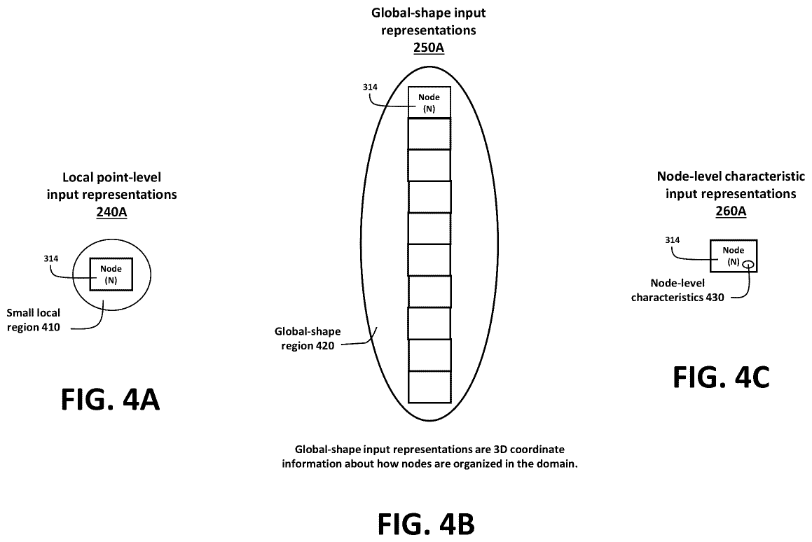

FIG. 4A depicts an example of how the local point-level input representations 240 (shown in FIG. 2) can be represented as local point-level input representations 240A of the inputs 238A (shown in FIG. 3). Known approaches to creating a generative model of any domain utilize machine learning techniques, which analyze the local node-level or point-level representations 240A of the domain. Each point-level representation 240A is local in that it provides information that is valid only for a relatively small local region 410 around the relevant point or node 314. Accordingly, known approaches to machine learning configure machine learning algorithms to “learn” patterns in local point-level input representations 240A of a domain by moving point-by-point through the nodes 314 regardless of whether the domain being modeled is 2D or 3D. Examples of the local point-level input representations 240A include strings and 2D graphs.

FIG. 4B depicts an example of how the global-shape input representations 250 (shown in FIG. 2) can be represented as global-shape input representations 250A of the inputs 238A (shown in FIG. 3). Embodiments of the invention address the shortcomings of using the local point-level input representations 240A and point-by-point learning to model 3D domains by including the use of 3D global-shape input representations 250A to generate the deep generative model 250 of a 3D domain. In contrast to the local point-level input representation 240A and point-by-point learning, the global-shape representation 250A provide information (global-shape region 420) that is valid for the entire domain, which is particularly beneficial when the domain is a 3D domain that include 3D shapes or geometries. It has been discovered by the inventors of the embodiments of the present invention that using global-shape input representations 250A that provide information (global-shape region 420) that is valid for the entire 3D domain to create the generative model 230 of the 3D domain improves how well the generative model 230 represents the domain's 3D information, which improves how well the model 230 represents (or reproduces) the domain's functionality.

FIG. 4C depicts an example of how the node-level characteristic input representations 260 (shown in FIG. 2) can be represented as node-level characteristic input representation 260A of the inputs 238A (shown in FIG. 3). In some embodiments of the invention, the inputs 238A (and the input representations 238) can be augmented by including the node-level characteristics 430 of the node 314. In accordance with aspects of the invention, including the node-level characteristics 430 of the 3D domain among the inputs 238A and/or the input representations 238 improve the ability of the resulting generative model 230 to represent or reproduce a wide range of the functionality of the 3D domain that is attributable to the node-level characteristics 230 that are present in the 3D domain.

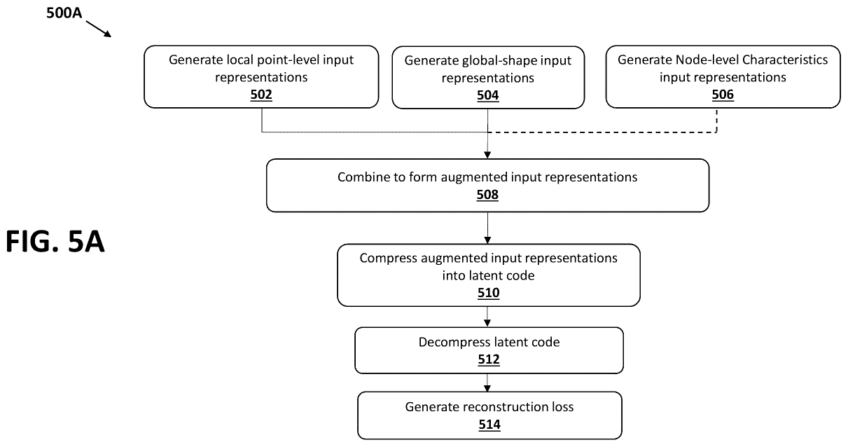

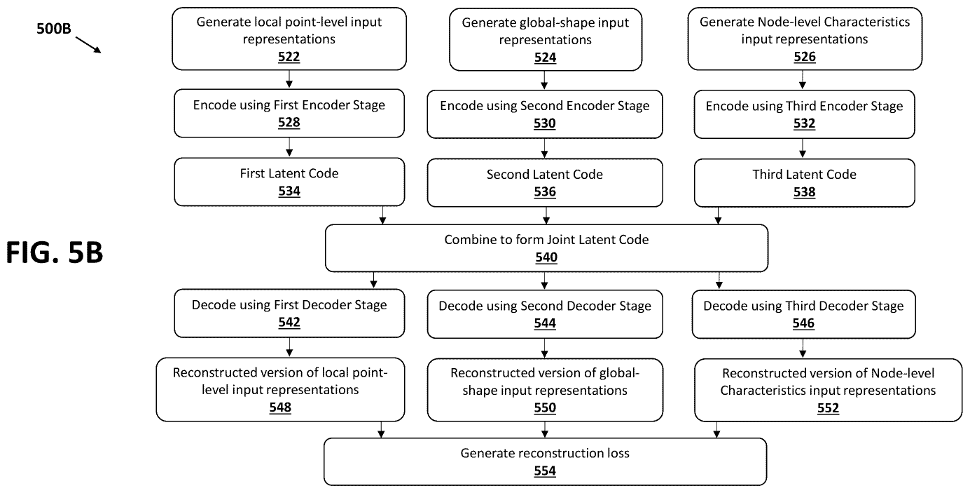

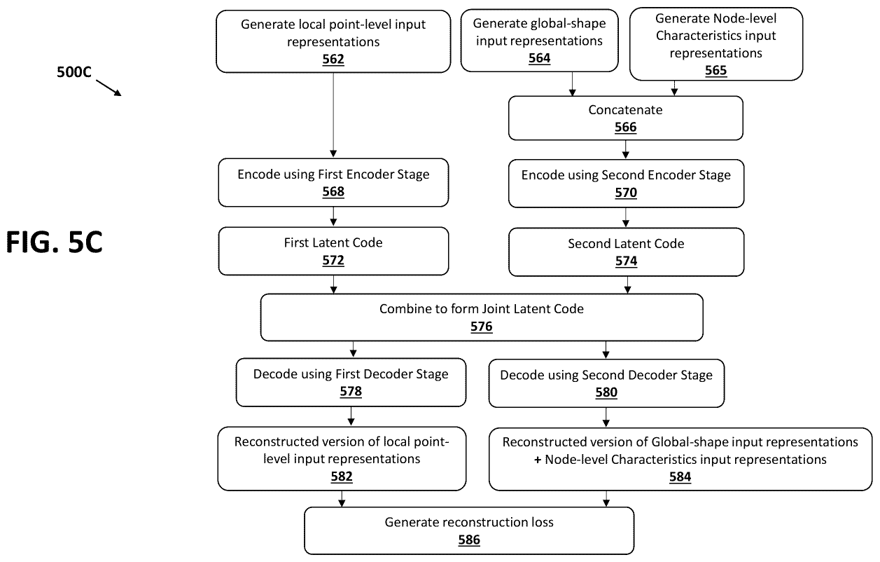

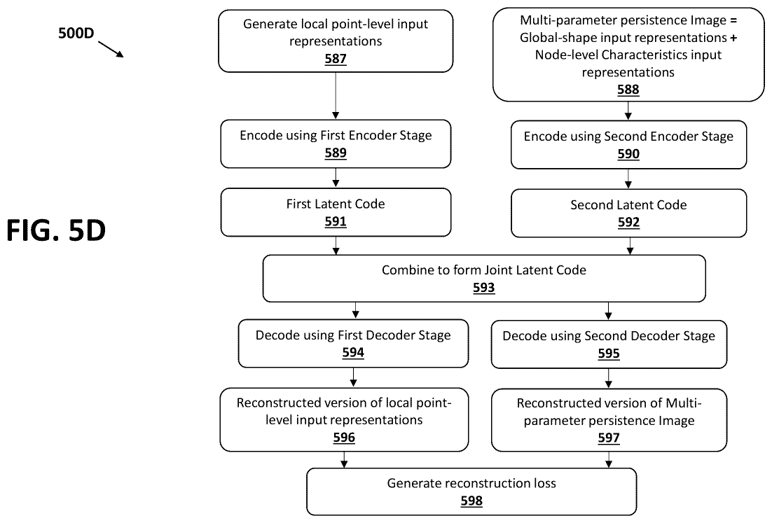

FIG. 5A depicts a flow diagram illustrating a methodology 500A in accordance with embodiments of the invention. The methodology 500A can be implemented by the system 200 (shown in FIG. 2) in accordance with aspects of the invention. At blocks 502, 504, 506, the processor 202 generates the local point-level input representations 240, the global-shape input representations 250, and the node-level characteristic input representations 260, and provides the same to block 508. At block 508, the methodology 500A combines the local point-level input representations 240, the global-shape input representations 250, and the node-level characteristic input representations 260 to form augmented input representations. At block 510, the methodology 500A compresses the augmented input representations into latent code. At block 512, the methodology 500A decompresses the latent code to generate reconstructed versions of the local point-level input representations 240, the global-shape input representations 250, and the node-level characteristic input representations 260. At block 514, the methodology 500A extracts a reconstruction loss from the difference between the local point-level input representations 240, the global-shape input representations 250, and the node-level characteristic input representations 260 generated at blocks 502, 504, 506 and the reconstructed versions of the local point-level input representations 240, the global-shape input representations 250, and the node-level characteristic input representations 260 generated at block 512.

FIG. 5B depicts a flow diagram illustrating a methodology 500B in accordance with embodiments of the invention. The methodology 500B can be implemented by the system 200 (shown in FIG. 2) in accordance with aspects of the invention. At blocks 522, 524, 526, the processor 202 generates the local point-level input representations 240, the global-shape input representations 250, and the node-level characteristic input representations 260, and provides the same to a first encoder stage at block 528, a second encoder stage at block 530, and a third encoder stage at block 532, respectively. At blocks 528, 530, 532, the methodology 500B encodes the respective inputs to generate a first latent code at block 534, a second latent code at block 536, and a third latent code at block 538. At block 540, the methodology 500B combines the first, second, and third latent codes to form a joint latent code. The methodology 500B decodes the joint latent code at block 542 using a first decoder stage. The methodology 500B decodes the joint latent code at block 544 using a second decoder stage. The methodology 500B decodes the joint latent code at block 546 using a third decoder stage. Block 548 uses the output from block 542 to generate a reconstructed version of the local point-level input representation 240. Block 550 uses the output from block 544 to generate a reconstructed version of the global-shape input representation 250. Block 552 uses the output from block 546 to generate a reconstructed version of the node-level characteristic input representation 260. At block 554, the methodology 500B generates a reconstruction loss based on a difference between the local point-level input representations 240, the global-shape input representations 250, and the node-level characteristic input representations 260 generated at blocks 522, 524, 526 and the reconstructed versions of the local point-level input representations 240, the global-shape input representations 250, and the node-level characteristic input representations 260 generated at blocks 548, 550, 552.

FIG. 5C depicts a flow diagram illustrating a methodology 500C in accordance with embodiments of the invention. The methodology 500C can be implemented by the system 200 (shown in FIG. 2) in accordance with aspects of the invention. At blocks 562, 564, 565, the processor 202 generates the local point-level input representations 240, the global-shape input representations 250, and the node-level characteristic input representations 260. The methodology 500C provide the local point-level input representations 240 to a first encoder stage at block 568. The methodology 500C concatenates the outputs from blocks 564, 565 at block 566 and provides its concatenated output to a second encoder stage at block 570. At block 568, the first encoder stage encodes the local point-level input representations 240 to generate the first latent code at block 572. At block 570, the second encoder stage encodes the concatenated output to generate the second latent code at block 574. At block 576, the methodology 500C combines the first and second latent codes to form a joint latent code. The methodology 500C decodes the joint latent code at block 578 using a first decoder stage. The methodology 500C decodes the joint latent code at block 580 using a second decoder stage. Block 582 uses the output from block 578 to generate a reconstructed version of the local point-level input representation 240. Block 584 uses the output from block 580 to generate a reconstructed version of the global-shape input representation 250 plus the node-level characteristic input representation 260. At block 586, the methodology 500C generates a reconstruction loss based on a difference between the local point-level input representations 240, the global-shape input representations 250, and the node-level characteristic input representations 260 generated at blocks 562, 564, 565 and the reconstructed versions of the local point-level input representations 240, the global-shape input representations 250, and the node-level characteristic input representations 260 generated at blocks 582, 584.

FIG. 5D depicts a flow diagram illustrating a methodology 500D in accordance with embodiments of the invention. The methodology 500D can be implemented by the system 200 (shown in FIG. 2) in accordance with aspects of the invention. At blocks 587, the processor 202 generates the local point-level input representations 240 and provides the same to a first encoder stage 589. At block 588, a multi-parameter persistence image is generated based on the global-shape input representations 250 and the node-level characteristic input representations 260. Block 588 provides the multi-parameter persistence image to a second encoder stage at block 590. At block 589, the methodology 500D encodes the local point-level input representation to generate the first latent code at block 591. At block 590, the methodology 500D encodes the multi-parameter persistence image to generate the second latent code at block 592. At block 593, the methodology 500D combines the first and second latent codes to form a joint latent code. The methodology 500D decodes the joint latent code at block 594 using a first decoder stage. The methodology 500D decodes the joint latent code at block 595 using a second decoder stage. Block 596 uses the output from block 594 to generate a reconstructed version of the local point-level input representation 240. Block 597 uses the output from block 595 to generate a reconstructed version of the multi-parameter persistence image, which is a reconstructed version of the global-shape input representation 250 plus the node-level characteristic input representation 260. At block 598, the methodology 500D generates a reconstruction loss based on a difference between the local point-level input representations 240, the global-shape input representations 250, and the node-level characteristic input representations 260 generated at blocks 587, 588 and the reconstructed versions of the local point-level input representations 240, the global-shape input representations 250, and the node-level characteristic input representations 260 generated at blocks 596, 597.

FIG. 6 depicts a diagram of a 3D molecular domain 100A that can be modeled using aspects of the invention. The 3D molecular domain 100A is not planar but is instead characterized by 3D structures. For example, methane has the shape of a regular tetrahedron with carbon at the center and a hydrogen atom at each corner. Thus, the 3D arrangement of the atoms 602 that constitute the molecular domain 100A is known as molecular geometry and includes the general shape of the molecule as well as bond lengths, bond angles, torsional angles and any other geometrical parameters that determine the position of each atom 602. The 3D molecular domain 100A includes molecular domain global-shape information 110A and molecular characteristics 120A (e.g., atomic charge, atomic weight, and the like).

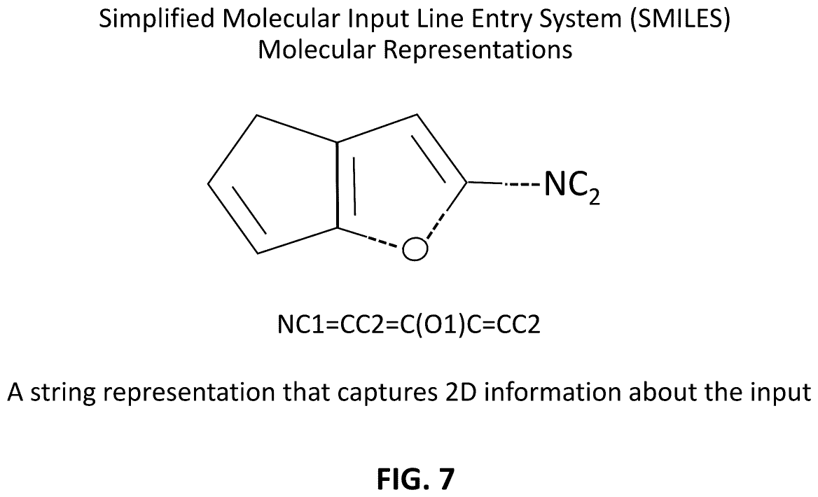

FIG. 7 depicts a simplified molecular input line entry system (SMILES) molecular representation in the form of a string representation that captures 2D information about the 3D molecular domain 100A. In accordance with aspects of the invention, although the string representation encodes information about the atoms 602 that are present in the 3D molecule domain 100A, the bonds between them, and some information about the substructures in the molecule, the string does not have any 3D information about how the atoms 602 are organized in the 3D molecular domain 100A. Thus, the string representation is an example of the local point-level (or node-level) input representation 240 (shown in FIG. 2).

FIG. 8 depicts diagrams 810, 820, 830 that illustrate the use of TDA analysis to generate the persistence image 830 that can be used as an example of the global-shape input representation 250 (shown in FIG. 2) that provides a global representation of the entire 3D molecular domain 100A. FIG. 8 shows an example of going from a 3D coordinate persistence diagram for homology dimension 1 (diagram 810) to a birth-persistence diagram 820 to a persistence image 830 for this same molecule. TDA uses tools from a branch of mathematics called “homology” or “homology theory” which is basically taking a look at the “shape of data.” TDA is a robust tool that counts the shapes (e.g., the number of holes) in the data at different dimensions. Persistent data shapes that are maintained throughout the topological data analysis are not just due to noise but are actually part of the data, and these holes or voids give you a sense of the shape of the data. TDA provides a rich representation of the relevant data, and the “persistence image” 830 takes the information extracted from the rich topological analysis and makes a vector out of it that you can be fed into a machine learning model. Persistence refers to the data that stays around such that it is probably represents a real feature of the data and not noise. The pixels in the image are the vector representation that captures the topological data representation of the 3D domain (e.g., the 3D molecular domain 100A).

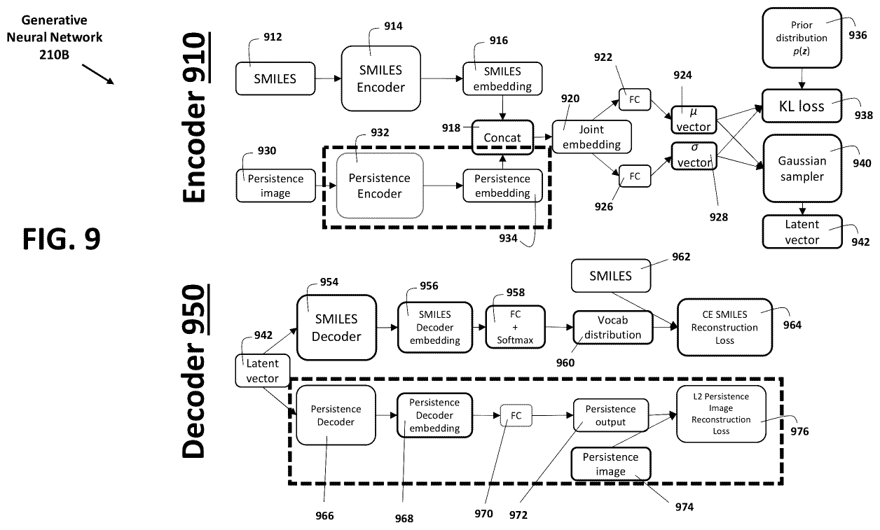

FIG. 9 depicts a GNN 210B in accordance with aspects of the invention. The GNN 210B is an example implementation of the GNN 210 (shown in FIG. 2). In accordance with embodiments of the invention, the GNN 210B is a VAE that includes an encoder 910 and a decoder 950. The encoder 910 includes a first encoder stage that processes SMILES string input representations 912, along with a second encoder stage that processes global-level persistence image input representations 930, which are an examples of the global-shape input representation 250. As shown, the first encoder stage encodes the SMILES input representations (generated at block 912) at block 914 to generate SMILES embeddings (or latent code) at block 916. The second encoder stage encodes the global-shape persistence image input representation at block 932 to generate persistence embeddings (or latent code) at block 934. Block 918 concatenates the SMILES embeddings and the persistence embeddings to generate joint embeddings at block 920. The joint embedding is fed into a high dimensional Gaussian distribution, represented by blocks 922, 926 to generate the random vectors 924, 928, which are then passed through blocks 936, 938, and 940 in a conventional manner to generate a latent vector 942. More specifically, the concatenated joint latent embedding coming from block 920 is copied and fed into two separate fully connected neural network layers (i.e., layers that perform matrix multiplication) represented in block 922 and block 926. The outputs of block 922 and block 926 are vectors that will be used to characterize a normal distribution's mean and variance, which are block 924 and block 928, respectively. In block 938, normal distribution is measured against a prior distribution coming from block 936 using the Kullback-Leibler (KL) divergence. The divergence calculated in block 938 is used as a signal to train the encoder 910. The vectors at block 924 and block 928 are also fed into a Gaussian sampler at block 940 which samples from the normal distribution defined by block 924 and block 928. The output of block 940 is the latent vector at block 942 that will be used in the decoder 950.

The decoder 950 has two separate paths. The latent vector at block 942 is copied and passed to each path. In block 954, the latent vector is decoded using a neural network into a vector (decoder embedding) at block 956, which is then passed through a fully connected (FC) layer and softmax non-linearity in block 958. The softmax function is used as the activation function in the output layer of neural network models that predict a multinomial probability distribution. That is, softmax is used as the activation function for multi-class classification problems where class membership is required on more than two class labels. The output generates at block 958 is the vector 960, which represents a probability distribution over the vocabulary of atoms present in the full training corpus of strings representing the molecules. The original string 962 (i.e., ground truth), along with a one-hot encoding of the same shape as the distribution vector 960 are passed to block 964, which calculates the cross-entropy (CE) loss. This CE reconstruction loss is used as a signal to train the encoder 910 and the decoder network 950. On the second path of the decoder 950, the latent vector 942 is passed to a persistence image decoder at block 966. The output of block 966 is block 968, which is a decoded vector representation of a molecule's persistence image. The output of block 968 is passed through another fully connected (FC) layer 970 which outputs the final decoded learned persistence image representation at block 972. The ground truth persistence image for the molecule at block 974 is passed to block 976 along with the output of block 972. In block 976 the Euclidean distance between the vectors generated at block 974 and block 972 is calculated. This distance is used as a signal to train the encoder 910 and the decoder 950.

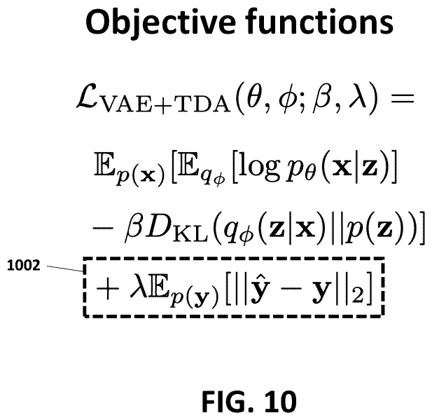

FIG. 10 depicts the objective functions that are used to train the variational autoencoder, and with the portion of the function added by the encoder and decoder segments that process the persistence image 930 is depicted at 1002.

FIG. 11 illustrates an example of a computer system 1100 that can be used to implement the computer-based components of the neural network system described herein. The computer system 1100 includes an exemplary computing device (“computer”) 1102 configured for performing various aspects of the content-based semantic monitoring operations described herein in accordance aspects of the invention. In addition to computer 1102, exemplary computer system 1100 includes network 1114, which connects computer 1102 to additional systems (not depicted) and can include one or more wide area networks (WANs) and/or local area networks (LANs) such as the Internet, intranet(s), and/or wireless communication network(s). Computer 1102 and additional system are in communication via network 1114, e.g., to communicate data between them.

Exemplary computer 1102 includes processor cores 1104, main memory (“memory”) 1110, and input/output component(s) 1112, which are in communication via bus 1103. Processor cores 1104 includes cache memory (“cache”) 1106 and controls 1108, which include branch prediction structures and associated search, hit, detect and update logic, which will be described in more detail below. Cache 1106 can include multiple cache levels (not depicted) that are on or off-chip from processor 1104. Memory 1110 can include various data stored therein, e.g., instructions, software, routines, etc., which, e.g., can be transferred to/from cache 1106 by controls 1108 for execution by processor 1104. Input/output component(s) 1112 can include one or more components that facilitate local and/or remote input/output operations to/from computer 1102, such as a display, keyboard, modem, network adapter, etc. (not depicted).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:
Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.
Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.
Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:
Private cloud: the cloud infrastructure is operated solely for an organization. It may be managed by the organization or a third party and may exist on-premises or off-premises.
Community cloud: the cloud infrastructure is shared by several organizations and supports a specific community that has shared concerns (e.g., mission, security requirements, policy, and compliance considerations). It may be managed by the organizations or a third party and may exist on-premises or off-premises.
Public cloud: the cloud infrastructure is made available to the general public or a large industry group and is owned by an organization selling cloud services.
Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Referring now to FIG. 12, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 12 are intended to be illustrative only and that computing nodes 10 and cloud computing environment 50 can communicate with any type of computerized device over any type of network and/or network addressable connection (e.g., using a web browser).

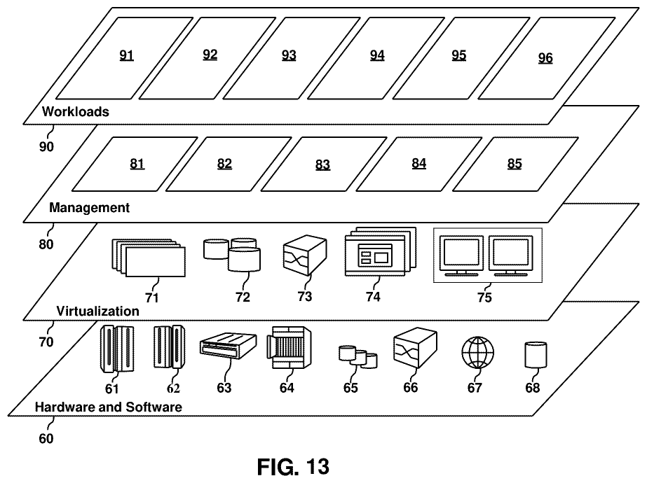

Referring now to FIG. 13, a set of functional abstraction layers provided by cloud computing environment 50 (FIG. 12) is shown. It should be understood in advance that the components, layers, and functions shown in FIG. 13 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and the generative neural network functionality 96.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
applying input representations of a three-dimensional (3D) domain to a generative neural network (GNN); and
using the GNN to form a generative model of the 3D domain based at least in part on the input representations;
wherein the input representations comprise a global-shape input representation of the 3D domain.

2. The computer-implemented method of claim 1, wherein the GNN is part of a cloud computing system.

3. The computer-implemented method of claim 1, wherein the global-shape input representation comprises a persistence image input representation.

4. The computer-implemented method of claim 1, wherein the input representations further comprise a local point-level input representation of the 3D domain.

5. The computer-implemented method of claim 4, wherein using the GNN to form the generative model of the 3D domain comprises:
encoding, using the GNN, the input representations to generate latent code;
decoding, using the GNN, the latent code to generate a reconstructed version of the input representations; and
generating, using the GNN, a reconstruction loss based at least in part on the reconstructed version of the input representations.

6. The computer-implemented method of claim 5, wherein:
the local point-level input representation comprises a string input representation; and
the global-shape input representation comprises a persistence image input representation.

7. The computer-implemented method of claim 5, wherein:
the input representations further comprise an input representation of a characteristic of the 3D domain;
the global-shape input representation comprises a first parameter of a multi-parameter persistence image; and
the input representation of the characteristic of the 3D domain comprises a second parameter of the multi-parameter persistence image.

8. A computer system comprising a memory and a processor communicatively coupled to the memory, wherein the processor is operable to perform operations comprising:
applying input representations of a three-dimensional (3D) domain to a generative neural network (GNN); and
using the GNN to form a generative model of the 3D domain based at least in part on the input representations;
wherein the input representations comprise a global-shape input representation of the 3D domain.

9. The computer system of claim 8, wherein the GNN is part of a cloud computing system.

10. The computer system of claim 8, wherein the global-shape input representation of the 3D domain comprises a persistence image.

11. The computer system of claim 8, wherein the input representations further comprise a local point-level input representation of the 3D domain.

12. The computer system of claim 11, wherein using the GNN to form the generative model of the 3D domain comprises:
encoding, using the GNN, the input representations to generate latent code;
decoding, using the GNN, the latent code to generate a reconstructed version of the input representations; and
generating, using the GNN, a reconstruction loss based at least in part on the reconstructed version of the input representations.

13. The computer system of claim 12, wherein:
the local point-level input representation comprises a string input representation; and
the global-shape input representation comprises a persistence image input representation.

14. The computer system of claim 12, wherein:
the input representations further comprise an input representation of a characteristic of the 3D domain;
the global-shape input representation of the 3D domain is represented as a first parameter of a multi-parameter persistence image; and
the input representation of the characteristic of the 3D domain is represented as a second parameter of the multi-parameter persistence image.

15. A computer program product comprising a computer readable storage medium having program instructions embodied therewith, the program instructions executable by a processor system to cause the processor system to perform operations comprising:
applying input representations of a three-dimensional (3D) domain to a generative neural network (GNN); and
using the GNN to form a generative model of the 3D domain based at least in part on the input representations;
wherein the input representations comprise a global-shape input representation of the 3D domain.

16. The computer program product of claim 15, wherein the GNN is part of a cloud computing system.

17. The computer program product of claim 15, wherein the global-shape input representation of the 3D domain comprises a persistence image.

18. The computer program product of claim 15, wherein the input representations further comprise a local point-level input representation of the 3D domain.

19. The computer program product of claim 18, wherein using the GNN to form the generative model of the 3D domain comprises:
encoding, using the GNN, the input representations to generate latent code;
decoding, using the GNN, the latent code to generate a reconstructed version of the input representations; and
generating, using the GNN, a reconstruction loss based at least in part on the reconstructed version of the input representations.

20. The computer program product of claim 19, wherein:
the local point-level input representation comprises a string input representation; and
the global-shape input representation comprises a persistence image input representation.

21. The computer program product of claim 19, wherein:
the input representations further comprise an input representation of a characteristic of the 3D domain;
the global-shape input representation of the 3D domain is represented as a first parameter of a multi-parameter persistence image; and
the input representation of the characteristic of the 3D domain is represented as a second parameter of the multi-parameter persistence image.

22. A computer system comprising a memory and a processor communicatively coupled to the memory, wherein the processor is operable to form a generative model of a three-dimensional (3D) domain by performing operations comprising:
encoding, using a generative neural network (GNN), input representations to generate latent code;
wherein the input representations comprise:
a string input representation of the 3D domain; and
a 3D coordinates input representation of the 3D domain;
decoding, using the GNN, the latent code to generate a reconstructed version of the input representations; and
generating, using the GNN, a reconstruction loss based at least in part on the reconstructed version of the input representations.

23. The computer system of claim 22, wherein:
the input representations further comprise an input representation of a characteristic of the 3D domain;
the 3D coordinates input representation of the 3D domain is represented as a first parameter of a multi-parameter persistence image; and
the input representation of the characteristic of the 3D domain is represented as a second parameter of the multi-parameter persistence image.

24. A computer system comprising a memory and a processor communicatively coupled to the memory, wherein the processor is operable to form a generative model of a three-dimensional (3D) domain by performing operations comprising:
encoding, using a generative neural network (GNN), input representations to generate latent code;
wherein the input representations comprise:
a string input representation of the 3D domain;
a 3D coordinates input representation of the 3D domain; and
an input representation of a characteristic of the 3D domain;
decoding, using the GNN, the latent code to generate a reconstructed version of the input representations; and
generating, using the GNN, a reconstruction loss based at least in part on the reconstructed version of the input representations.

25. The computer system of claim 24, wherein:
the 3D domain comprises a molecule; and
the characteristic of the molecule is selected from the group consisting of an atomic charge and an atomic weight.
